(19) United States
(12) Patent Application Publication
(10) Pub. No.: US 2018/0031715 A1
(43) Pub. Date: Feb. 1, 2018

(54) RADIOGRAPHY SYSTEM, RADIOGRAPHY METHOD, AND RADIOGRAPHY PROGRAM

(71) Applicant: FUJIFILM Corporation, Tokyo (JP)

(72) Inventor: Takeshi KUWABARA, Kanagawa (JP)

(21) Appl. No.: 15/647,266

(22) Filed: Jul. 12, 2017

(30) Foreign Application Priority Data
Jul. 29, 2016 (JP) 2016-150591

(51) Int. Cl. G01T 1/20; G01T 7/00; A61B 6/00

(52) U.S. Cl. CPC G01T 1/2018; A61B 6/505; A61B 6/4233; G01T 7/00

(57) ABSTRACT

A radiography system includes: a radiography apparatus including a first radiation detector and a second radiation detector which is provided so as to be stacked on the side of the first radiation detector from which the radiation is transmitted and emitted; and an integrated control unit that specifies a predetermined time related to the emission of the radiation, on the basis of a first detection result that is a detection result of a predetermined time related to the emission of the radiation using a first electric signal output from the first radiation detector and a second detection result that is a detection result of a predetermined time related to the emission of the radiation using a second electric signal output from the second radiation detector.

### CROSS-REFERENCE TO RELATED APPLICATIONS

The present application claims priority under 35 U.S.C §119 to Japanese Patent Application No. 2016-150591, filed on Jul. 29, 2016, which is hereby expressly incorporated by reference, in its entirety, into the present application.

### BACKGROUND

### Technical Field

The present disclosure relates to a radiography system, a radiography method, and a radiography program.

### Related Art

For example, as disclosed in WO2013/047193A, a radiography apparatus has been known that comprises two radiation detectors each of which includes a plurality of pixels that accumulate charge corresponding to the amount of radiation emitted and which are provided so as to be stacked.

In addition, a technique has been known which detects a predetermined time related to the emission of radiation, such as the time when the emission of radiation starts and the time when the emission of radiation ends, on the basis of an electric signal of which the level generally increases as the amount of charge output from each pixel of a radiation detector of a radiography apparatus increases.

However, in a case in which radiographic images are captured by two radiation detectors disclosed in, for example, WO2013/047193A, radiation that has been transmitted through the radiation detector provided on the incident side of the radiation reaches the radiation detector provided on the emission side of the radiation. Therefore, the amount of radiation that reaches the radiation detector provided on the emission side of the radiation is less than the amount of radiation that reaches the radiation detector provided on the incident side and the amount of radiation used to generate a radiographic image is reduced.

Therefore, in the radiation detector provided on the incident side of the radiation and the radiation detector provided on the emission side of the radiation, the detection results of the predetermined time related to the emission of radiation are different from each other. As a result, in some cases, it is difficult to appropriately detect the emission of radiation in the entire radiography apparatus.

### SUMMARY

The present disclosure has been made in view of the above-mentioned problems and an object of the present disclosure is to provide a technique that can appropriately detect the emission of radiation even when the amount of radiation emitted to a second radiation detector is less than the amount of radiation emitted to a first radiation detector.

In order to achieve the object, according to an aspect of the invention, there is provided a radiography system comprising: a radiography apparatus comprising a first radiation detector in which a plurality of pixels, each of which includes a conversion element that generates a larger amount of charge as it is irradiated with a larger amount of radiation, are two-dimensionally arranged and a second radiation detector which is provided so as to be stacked on a side of the first radiation detector from which the radiation is transmitted and emitted and in which a plurality of pixels, each of which includes a conversion element that generates a larger amount of charge as it is irradiated with a larger amount of radiation, are two-dimensionally arranged; and a specification unit that specifies a predetermined time related to the emission of the radiation, on the basis of a first detection result that is a detection result of a predetermined time related to the emission of the radiation using a first electric signal which is obtained by converting charge generated in the pixels of the first radiation detector and of which the level increases as the amount of charge increases and a second detection result that is a detection result of a predetermined time related to the emission of the radiation using a second electric signal which is obtained by converting charge generated in the pixels of the second radiation detector and of which the level increases as the amount of charge increases.

In the radiography system according to the present disclosure, in a case in which the first detection result and the second detection result are different from each other, the specification unit may specify the predetermined time related to the emission of the radiation on the basis of a predetermined detection result of the first and second detection results.

In the radiography system according to the present disclosure, the predetermined detection result may be the first detection result.

The radiography system according to the present disclosure may further comprise a detection result setting unit that sets the predetermined detection result.

In the radiography system according to the present disclosure, the specification unit may further specify whether to continue to perform an operation of accumulating charge in the plurality of pixels of the first radiation detector and an operation of accumulating charge in the plurality of pixels of the second radiation detector, using a first noise detection result which is a detection result of noise included in the first electric signal and a second noise detection result which is a detection result of noise included in the second electric signal after the operation of accumulating charge in the plurality of pixels of the first radiation detector and the operation of accumulating charge in the plurality of pixels of the second radiation detector start.

In the radiography system according to the present disclosure, the first noise detection result and the second noise detection result that the specification unit uses to specify whether to continue to perform the operation of accumulating charge in the plurality of pixels of the first radiation detector and the operation of accumulating charge in the plurality of pixels of the second radiation detector may be a detection result of noise included in the first electric signal using the first electric signal and a detection result of noise included in the second electric signal using the second electric signal, respectively.

The radiography system according to the present disclosure may further comprise: a first detection unit that detects at least one of an impact or an electromagnetic wave which is applied from the outside to the first radiation detector; and a second detection unit that detects at least one of an impact or an electromagnetic wave which is applied from the outside to the second radiation detector. The first noise detection result and the second noise detection result that the specification unit uses to specify whether to continue to perform the operation of accumulating charge in the plurality of pixels of the first radiation detector and the operation of accumulating charge in the plurality of pixels of the second radiation detector may be a detection result of noise included in the first electric signal using a detection result of the first detection unit and a detection result of noise included in the second electric signal using a detection result of the second detection unit, respectively.

In the radiography system according to the present disclosure, in a case in which the first noise detection result and the second noise detection result are different from each other, the specification unit may specify whether to continue to perform the operation of accumulating charge in the plurality of pixels of the first radiation detector and the operation of accumulating charge in the plurality of pixels of the second radiation detector, using a predetermined noise detection result of the first and second noise detection results.

In the radiography system according to the present disclosure, the predetermined noise detection result may be the first noise detection result.

The radiography system according to the present disclosure may further comprise a noise detection result setting unit that sets the predetermined noise detection result.

In the radiography system according to the present disclosure, in a case in which at least one of the first noise detection result or the second noise detection result indicates that noise has been detected, the specification unit may specify to stop the operation of accumulating charge in the plurality of pixels of the first radiation detector and the operation of accumulating charge in the plurality of pixels of the second radiation detector.

In the radiography system according to the present disclosure, the specification unit may specify a time when the emission of the radiation starts as the predetermined time related to the emission of the radiation.

In the radiography system according to the present disclosure, the radiography apparatus may further comprise the specification unit.

In the radiography system according to the present disclosure, each of the first radiation detector and the second radiation detector may comprise a light emitting layer that is irradiated with radiation and emits light. The plurality of pixels of each of the first radiation detector and the second radiation detector may receive the light, generate the charge, and accumulate the charge. The light emitting layer of the first radiation detector and the light emitting layer of the second radiation detector may have different compositions.

In the radiography system according to the present disclosure, the light emitting layer of the first radiation detector may include CsI and the light emitting layer of the second radiation detector may include GOS.

The radiography system according to the present disclosure may further comprise a derivation unit that derives at least one of bone mineral content or bone density, using a first radiographic image captured by the first radiation detector and a second radiographic image captured by the second radiation detector.

In order to achieve the object, according to another aspect of the present disclosure, there is provided a radiography method that is performed by a radiography apparatus comprising a first radiation detector in which a plurality of pixels, each of which includes a conversion element that generates a larger amount of charge as it is irradiated with a larger amount of radiation, are two-dimensionally arranged and a second radiation detector which is provided so as to be stacked on a side of the first radiation detector from which the radiation is transmitted and emitted and in which a plurality of pixels, each of which includes a conversion element that generates a larger amount of charge as it is irradiated with a larger amount of radiation, are two-dimensionally arranged. The radiography method comprises specifying a predetermined time related to the emission of the radiation, on the basis of a first detection result that is a detection result of a predetermined time related to the emission of the radiation using a first electric signal which is obtained by converting charge generated in the pixels of the first radiation detector and of which the level increases as the amount of charge increases and a second detection result that is a detection result of a predetermined time related to the emission of the radiation using a second electric signal which is obtained by converting charge generated in the pixels of the second radiation detector and of which the level increases as the amount of charge increases.

In order to achieve the object, according to still another aspect of the present disclosure, there is provided a radiography program that causes a computer controlling a radiography apparatus comprising a first radiation detector in which a plurality of pixels, each of which includes a conversion element that generates a larger amount of charge as it is irradiated with a larger amount of radiation, are two-dimensionally arranged and a second radiation detector which is provided so as to be stacked on a side of the first radiation detector from which the radiation is transmitted and emitted and in which a plurality of pixels, each of which includes a conversion element that generates a larger amount of charge as it is irradiated with a larger amount of radiation, are two-dimensionally arranged to perform specifying a predetermined time related to the emission of the radiation, on the basis of a first detection result that is a detection result of a predetermined time related to the emission of the radiation using a first electric signal which is obtained by converting charge generated in the pixels of the first radiation detector and of which the level increases as the amount of charge increases and a second detection result that is a detection result of a predetermined time related to the emission of the radiation using a second electric signal which is obtained by converting charge generated in the pixels of the second radiation detector and of which the level increases as the amount of charge increases.

According to the present disclosure, it is possible to appropriately detect the emission of radiation even when the amount of radiation emitted to the second radiation detector is less than the amount of radiation emitted to the first radiation detector.

### BRIEF DESCRIPTION OF THE DRAWINGS

FIG. 1 is a block diagram illustrating an example of the structure of a radiography system according to an embodiment.

FIG. 2 is a side cross-sectional view illustrating an example of the structure of a radiography apparatus according to this embodiment.

FIG. 3 is a block diagram illustrating an example of the structure of a main portion of an electric system of the radiography apparatus according to this embodiment.

FIG. 4 is a circuit diagram illustrating an example of the structure of a signal processing unit according to this embodiment.

FIG. 5 is a block diagram illustrating an example of the structure of a main portion of an electric system of a console according to this embodiment.

FIG. 6 is a graph illustrating the amount of radiation that reaches each of a first radiation detector and a second radiation detector according to this embodiment.

FIG. 7 is a flowchart illustrating an example of the flow of an overall imaging process according to this embodiment.

FIG. 8 is a flowchart illustrating an example of the flow of an image generation process in the overall imaging process according to this embodiment.

FIG. 9 is a front view schematically illustrating a bone tissue region and a soft tissue region according to this embodiment.

FIG. 10 is a flowchart illustrating an example of the flow of an imaging control process according to this embodiment.

FIG. 11 is a diagram schematically illustrating an example of a selection screen for selecting a detection result having priority.

FIG. 12 is a flowchart illustrating an example of the flow of a first imaging process and a second imaging process according to this embodiment.

FIG. 13 is a diagram schematically illustrating a variation in the amount of radiation emitted from a radiation source over an irradiation time.

FIG. 14 is a diagram schematically illustrating an example of a selection screen for selecting a noise detection result having priority.

FIG. 15 is a block diagram illustrating another example of the structure of the main portion of the electric system of the radiography apparatus according to this embodiment.

### DETAILED DESCRIPTION

Hereinafter, an embodiment of the invention will be described in detail with reference to the drawings.

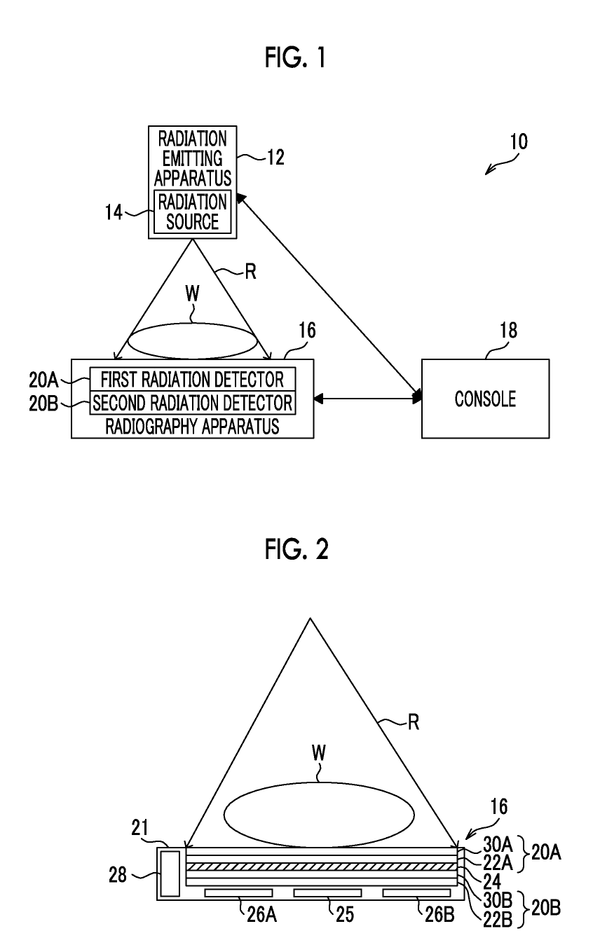

First, the structure of a radiography system 10 according to this embodiment will be described with reference to FIG. 1. As illustrated in FIG. 1, the radiography system 10 comprises a radiation emitting apparatus 12, a radiography apparatus 16, and a console 18. The console 18 according to this embodiment is an example of an image processing apparatus according to the invention.

The radiation emitting apparatus 12 according to this embodiment comprises a radiation source 14 that irradiates a subject W, which is an example of an imaging target, with radiation R such as X-rays. An example of the radiation emitting apparatus 12 is a treatment cart. A method for instructing the radiation emitting apparatus 12 to emit the radiation R is not particularly limited. For example, in a case in which the radiation emitting apparatus 12 comprises an irradiation button, a user, such as a doctor or a radiology technician, may press the irradiation button to instruct the emission of the radiation R such that the radiation R is emitted from the radiation emitting apparatus 12. In addition, for example, the user may operate the console 18 to instruct the emission of the radiation R such that the radiation R is emitted from the radiation emitting apparatus 12.

When receiving a command to start the emission of the radiation R, the radiation emitting apparatus 12 emits the radiation R from the radiation source 14 according to emission conditions, such as a tube voltage, a tube current, and an irradiation period.

The radiography apparatus 16 according to this embodiment comprises a first radiation detector 20A and a second radiation detector 20B that detect the radiation R which has been emitted from the radiation emitting apparatus 12 and then transmitted through the subject W. The radiography apparatus 16 captures radiographic images of the subject W using the first radiation detector 20A and the second radiation detector 20B. Hereinafter, in a case in which the first radiation detector 20A and the second radiation detector 20B do not need to be distinguished from each other, they are generically referred to as “radiation detectors 20”.

Next, the structure of the radiography apparatus 16 according to this embodiment will be described with reference to FIG. 2. As illustrated in FIG. 2, the radiography apparatus 16 comprises a plate-shaped housing 21 that transmits the radiation R and has a waterproof, antibacterial, and airtight structure. The housing 21 includes the first radiation detector 20A, the second radiation detector 20B, a radiation limitation member 24, a control board 25, a control board 26A, a control board 26B, and a case 28.

The first radiation detector 20A is provided on the incident side of the radiation R and the second radiation detector 20B is provided so as to be stacked on the side of the first radiation detector 20A from which the radiation R is transmitted and emitted in the radiography apparatus 16. The first radiation detector 20A comprises a thin film transistor (TFT) substrate 30A and a scintillator 22A which is an example of a light emitting layer that is irradiated with the radiation R and emits light corresponding to the amount of radiation R emitted. The TFT substrate 30A and the scintillator 22A are stacked in the order of the TFT substrate 30A and the scintillator 22A from the incident side of the radiation R. The term “stacked” means a state in which the first radiation detector 20A and the second radiation detector 20B overlap each other in a case in which the first radiation detector 20A and the second radiation detector 20B are seen from the incident side or the emission side of the radiation R in the radiography apparatus 16 and it does not matter how they overlap each other. For example, the first radiation detector 20A and the second radiation detector 20B, or the first radiation detector 20A, the radiation limitation member 24, and the second radiation detector 20B may overlap while coming into contact with each other or may overlap with a gap therebetween in the stacking direction.

The second radiation detector 20B comprises a TFT substrate 30B and a scintillator 22B which is an example of the light emitting layer. The TFT substrate 30B and the scintillator 22B are stacked in the order of the TFT substrate 30B and the scintillator 22B from the incident side of the radiation R.

That is, the first radiation detector 20A and the second radiation detector 20B are so-called irradiation side sampling (ISS) radiation detectors that are irradiated with the radiation R from the side of the TFT substrates 30A and 30B.

In the radiography apparatus 16 according to this embodiment, the scintillator 22A of the first radiation detector 20A and the scintillator 22B of the second radiation detector 20B have different compositions. Specifically, for example, the scintillator 22A includes CsI (Tl) (cesium iodide having thallium added thereto) as a main component and the scintillator 22B includes gadolinium oxysulfide (GOS) as a main component. GOS has a higher sensitivity to the high-energy radiation R than CsI. In addition, a combination of the composition of the scintillator 22A and the composition of the scintillator 22B is not limited to the above-mentioned example and may be a combination of other compositions or a combination of the same compositions.

The radiation limitation member 24 that limits the transmission of the radiation R is provided between the first radiation detector 20A and the second radiation detector 20B. An example of the radiation limitation member 24 is a metal plate made of, for example, copper or tin. It is preferable that a variation in the thickness of the radiation limitation member 24 in the incident direction of the radiation R is equal to or less than 1% in order to uniformize limitations (transmissivity) on the radiation.

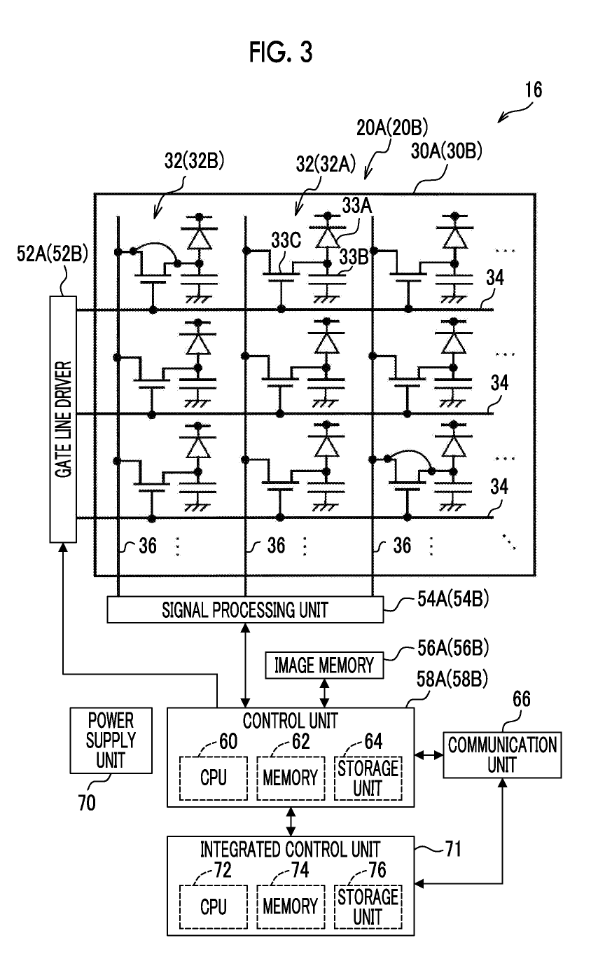

An electronic circuit, such as an integrated control unit 71 (see FIG. 3) which will be described below, is formed on the control board 25. The control board 26A is provided so as to correspond to the first radiation detector 20A and electronic circuits, such as an image memory 56A and a control unit 58A which will be described below, are formed on the control board 26A. The control board 26B is provided so as to correspond to the second radiation detector 20B and electronic circuits, such as an image memory 56B and a control unit 58B which will be described below, are formed on the control board 26B. The control board 25, the control board 26A, and the control board 26B are provided on the side of the second radiation detector 20B which is opposite to the incident side of the radiation R.

As illustrated in FIG. 2, the case 28 is provided at a position (that is, outside the range of an imaging region) that does not overlap the radiation detector 20 at one end of the housing 21. For example, a power supply unit 70 which will be described below is accommodated in the case 28. The installation position of the case 28 is not particularly limited. For example, the case 28 may be provided at a position that overlaps the radiation detector 20 on the side of the second radiation detector 20B which is opposite to the incident side of the radiation R.

Next, the structure of a main portion of an electric system of the radiography apparatus 16 according to this embodiment will be described with reference to FIG. 3.

As illustrated in FIG. 3, a plurality of pixels 32 are two-dimensionally provided in one direction (a row direction in FIG. 3) and an intersection direction (a column direction in FIG. 3) that intersects the one direction on the TFT substrate 30A.

In this embodiment, among the plurality of pixels 32, pixels 32A for capturing a radiographic image and pixels 32B for detecting radiation are predetermined. The pixel 32A for capturing a radiographic image is a pixel 32 that detects the radiation R and is used to generate an image indicated by the radiation R. The pixel 32B for detecting radiation is a pixel 32 that is used to detect, for example, the start of the emission of the radiation R and outputs charge even for a charge accumulation period (which will be described in detail below).

The pixel 32 includes a sensor unit 33A, a capacitor 33B, and a field effect thin film transistor (TFT; hereinafter, simply referred to as a “thin film transistor”) 33C. The sensor unit 33A according to this embodiment is an example of a conversion element according to the invention. In the pixel 32A for capturing a radiographic image and the pixel 32B for detecting radiation, the thin film transistors 33C have different structures.

The sensor unit 33A includes, for example, an upper electrode, a lower electrode, and a photoelectric conversion film which are not illustrated, absorbs the light emitted from the scintillator 22A, and generates charge. The capacitor 33B accumulates the charge generated by the sensor unit 33A. The thin film transistor 33C of the pixel 32A for capturing a radiographic image reads the charge accumulated in the capacitor 33B and outputs the charge in response to a control signal. In contrast, the thin film transistor 33C of the pixel 32B for detecting radiation has a source and drain which are short-circuited. Therefore, in the pixel 32B for detecting radiation, the charge generated by the sensor unit 33A flows to a data line 36, regardless of the switching state of the thin film transistor 33C.

According to this structure, as the pixel 32 according to this embodiment is irradiated with a larger amount of radiation, a larger amount of charge is accumulated in the pixel 32.

A plurality of gate lines 34 which extend in the one direction and are used to turn on and off each thin film transistor 33C are provided on the TFT substrate 30A. In addition, a plurality of data lines 36 which extend in the intersection direction and to which the charge read by the thin film transistors 33C in an on state is output are provided on the TFT substrate 30A.

A gate line driver 52A is provided on one side of two adjacent sides of the TFT substrate 30A and a signal processing unit 54A is provided on the other side. Each gate line 34 of the TFT substrate 30A is connected to the gate line driver 52A and each data line 36 of the TFT substrate 30A is connected to the signal processing unit 54A.

The thin film transistors 33C corresponding to each gate line 34 on the TFT substrate 30A are sequentially turned on (in units of row illustrated in FIG. 3 in this embodiment) by control signals which are supplied from the gate line driver 52A through the gate lines 34. The charge which is read by the thin film transistor 33C of the pixel 32A for capturing a radiographic image in an on state is transmitted as an electric signal through the data line 36 and is input to the signal processing unit 54A. In this way, charge is sequentially read from each gate line 34 (in units of row illustrated in FIG. 3 in this embodiment) and image data indicating a two-dimensional radiographic image is generated by the signal processing unit 54A. In addition, the charge which is read by the thin film transistor 33C of the pixel 32B for detecting radiation is transmitted as an electric signal through the data line 36 and is input to the signal processing unit 54A. However, image data indicating a radiographic image is not generated and the charge is output to the control unit 58A.

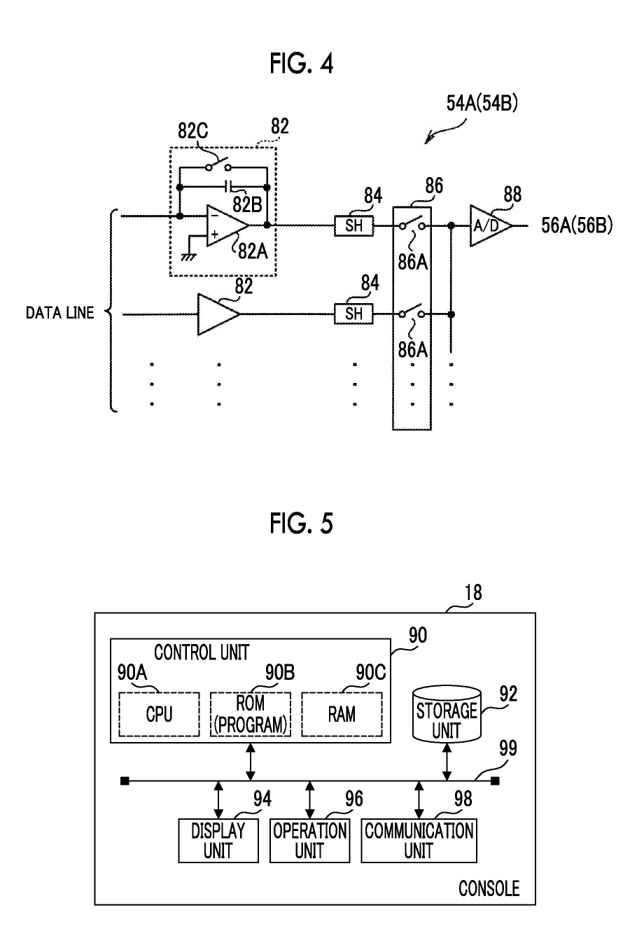

As illustrated in FIG. 4, the signal processing unit 54A comprises a variable gain pre-amplifier (charge amplifier) 82 and a sample-and-hold circuit 84 which correspond to each data line 36.

The variable gain pre-amplifier 82 includes an operational amplifier 82A that has a positive input side grounded and a capacitor 82B and a reset switch 82C that are connected in parallel to each other between a negative input side and an output side of the operational amplifier 82A. The reset switch 82C is turned on and off by the control unit 58A.

In addition, the signal processing unit 54A according to this embodiment comprises a multiplexer 86 and an analog/digital (A/D) converter 88. The sampling time of the sample-and-hold circuit 84 and the turn-on and turn-off of a switch 86A provided in the multiplexer 86 are controlled by the control unit 58A.

When a radiographic image is detected, first, the control unit 58A maintains the reset switch 82C of the variable gain pre-amplifier 82 in an on state for a predetermined period to release the charge accumulated in the capacitor 82B.

In contrast, the charge generated in the pixel 32B for detecting radiation due to irradiation with the radiation R is read to the data line 36 by the thin film transistor 33C, regardless of the switching state of the thin film transistor 33C. In addition, the charge generated in the pixel 32A for capturing a radiographic image is accumulated in the capacitor 33B and is read to the data line 36 by the thin film transistor 33C in an on state. The charge read to the data line 36 is transmitted as an electric signal and is then amplified by the corresponding variable gain pre-amplifier 82 at a predetermined gain.

After the above-mentioned discharging is performed, the control unit 58A drives the sample-and-hold circuit 84 for a predetermined period such that the level of the electric signal amplified by the variable gain pre-amplifier 82 is held and sampled by the sample-and-hold circuit 84.

Then, the signal levels sampled by each sample-and-hold circuit 84 are sequentially selected by the multiplexer 86 and are then converted into digital signal levels by the A/D converter 88 under the control of the control unit 58A. In this way, image data indicating the captured radiographic image is acquired. Hereinafter, the digital signal obtained by converting the electric signal (first electric signal) using the A/D converter 88 in the signal processing unit 54A is referred to as a “first digital signal” and the digital signal obtained by converting the electric signal (second electric signal) using the A/D converter 88 in the signal processing unit 54B is referred to as a “second digital signal”. In addition, in a case in which the first digital signal and the second digital signal do not need to be distinguished from each other, they are generically referred to as “digital signals”.

The control unit 58A which will be described below is connected to the signal processing unit 54A. The image data output from the A/D converter of the signal processing unit 54A is sequentially output the control unit 58A. The image memory 56A is connected to the control unit 58A. The image data sequentially output from the signal processing unit 54A is sequentially stored in the image memory 56A under the control of the control unit 58A. The image memory 56A has memory capacity that can store a predetermined amount of image data. Whenever a radiographic image is captured, captured image data is sequentially stored in the image memory 56A.

The control unit 58A comprises a central processing unit (CPU) 60, a memory 62 including, for example, a read only memory (ROM) and a random access memory (RAM), and a non-volatile storage unit 64 such as a flash memory. An example of the control unit 58A is a microcomputer.

The control unit 58A according to this embodiment has a function that outputs a first detection result, which is the detection result of the time when the emission of the radiation R has started, to the integrated control unit 71 according to whether the value of the first digital signal is equal to or greater than a predetermined start threshold value, which will be described in detail below. In some cases, the control unit 58A according to this embodiment erroneously detects the time when the emission of the radiation R has started, on the basis of charge that is generated as noise due to disturbance, such as impact and electromagnetic waves, particularly, vibration. Therefore, the control unit 58A according to this embodiment has a function that outputs a first noise detection result, which is the detection result of the generation of noise using the first digital signal, to the integrated control unit 71, which will be described in detail below.

The integrated control unit 71 comprises a CPU 72, a memory 74 including, for example, a ROM and a RAM, and a non-volatile storage unit 76 such as a flash memory. An example of the integrated control unit 71 is a microcomputer. The control unit 58A and the integrated control unit 71 are connected such that they can communicate with each other.

The integrated control unit 71 according to this embodiment has a function that specifies the time when the emission of the radiation R has started, using a predetermined result with priority of the first detection result and a second detection result output from the control unit 58A, which will be described in detail below. In addition, the integrated control unit 71 according to this embodiment has a function that controls the control unit 58A and the control unit 58B such that the accumulation of charge in each pixel 32 is stopped in a case in which at least one of the first noise detection result or a second noise detection result indicates that the generation of noise has been detected, which will be described in detail below.

A communication unit 66 is connected to the control unit 58A and the integrated control unit 71 and transmits and receives various kinds of information to and from external apparatuses, such as the radiation emitting apparatus 12 and the console 18, using at least one of wireless communication or wired communication. The power supply unit 70 supplies power to each of the above-mentioned various circuits or elements (for example, the gate line driver 52A, the signal processing unit 54A, the image memory 56A, the control unit 58A, the communication unit 66, and the integrated control unit 71). In FIG. 3, lines for connecting the power supply unit 70 to various circuits or elements are not illustrated in order to avoid complication.

Components of the TFT substrate 30B, the gate line driver 52B, the signal processing unit 54B, the image memory 56B, and the control unit 58B of the second radiation detector 20B have the same structures as the corresponding components of the first radiation detector 20A and thus the description thereof will not be repeated here.

The control unit 58B according to this embodiment has a function that outputs the second detection result, which is the detection result of the time when the emission of the radiation R has started, to the integrated control unit 71 according to whether the value of the second digital signal is equal to or greater than a predetermined start threshold value, which will be described in detail below. In addition, the control unit 58B according to this embodiment has a function that outputs the second noise detection result, which is the detection result of the generation of noise using the second digital signal, to the integrated control unit 71, which will be described in detail below.

The control unit 58A and the control unit 58B are connected such that they can communicate with each other.

According to the above-mentioned structure, the radiography apparatus 16 according to this embodiment captures radiographic images using the first radiation detector 20A and the second radiation detector 20B.

Next, the structure of the console 18 according to this embodiment will be described with reference to FIG. 5. As illustrated in FIG. 5, the console 18 comprises a control unit 90. The control unit 90 comprises a CPU 90A that controls the overall operation of the console 18, a ROM 90B in which, for example, various programs or various parameters are stored in advance, and a RAM 90C that is used as, for example, a work area when the CPU 90A executes various programs.

In addition, the console 18 comprises a non-volatile storage unit 92 such as a hard disk drive (HDD). The storage unit 92 stores and holds image data indicating a radiographic image captured by the first radiation detector 20A, image data indicating a radiographic image captured by the second radiation detector 20B, and various other data. Hereinafter, the radiographic image captured by the first radiation detector 20A is referred to as a “first radiographic image” and image data indicating the first radiographic image is referred to as “first radiographic image data”. In addition, hereinafter, the radiographic image captured by the second radiation detector 20B is referred to as a “second radiographic image” and image data indicating the second radiographic image is referred to as “second radiographic image data”. In a case in which the “first radiographic image” and the “second radiographic image” are generically named, they are simply referred to as “radiographic images”.

The console 18 further comprises a display unit 94, an operation unit 96, and a communication unit 98. The display unit 94 displays, for example, information related to imaging and a captured radiographic image. The user uses the operation unit 96 to input, for example, a command to capture a radiographic image and a command related to image processing for a captured radiographic image. For example, the operation unit 96 may have the form of a keyboard or may have the form of a touch panel that is integrated with the display unit 94. The communication unit 98 transmits and receives various kinds of information to and from the radiation emitting apparatus 12 and the radiography apparatus 16, using at least one of wireless communication or wired communication. In addition, the communication unit 98 transmits and receives various kinds of information to and from external systems, such as a picture archiving and communication system (PACS) and a radiology information system (RIS), using at least one of wireless communication or wired communication.

The control unit 90, the storage unit 92, the display unit 94, the operation unit 96, and the communication unit 98 are connected to each other through a bus 99.

As described above, in the radiography apparatus 16 according to this embodiment, the amount of radiation that reaches the second radiation detector 20B is less than the amount of radiation that reaches the first radiation detector 20A. In addition, the radiation limitation member 24 generally has the characteristic that it absorbs a larger number of low-energy components than high-energy components in energy forming the radiation R, which depends on the material forming the radiation limitation member 24. Therefore, the energy distribution of the radiation R that reaches the second radiation detector 20B has a larger number of high-energy components than the energy distribution of the radiation R that reaches the first radiation detector 20A.

In this embodiment, for example, about 50% of the radiation R that has reached the first radiation detector 20A is absorbed by the first radiation detector 20A and is used to capture a radiographic image. In addition, about 60% of the radiation R that has passed through the first radiation detector 20A and reached the radiation limitation member 24 is absorbed by the radiation limitation member 24. About 50% of the radiation R that has passed through the first radiation detector 20A and the radiation limitation member 24 and reached the second radiation detector 20B is absorbed by the second radiation detector 20B and is used to capture a radiographic image.

That is, the amount of radiation (the amount of charge generated by the second radiation detector 20B) used to capture a radiographic image by the second radiation detector 20B is about 20% of the amount of radiation used to capture a radiographic image by the first radiation detector 20A. In addition, the ratio of the amount of radiation used to capture a radiographic image by the second radiation detector 20B to the amount of radiation used to capture a radiographic image by the first radiation detector 20A is not limited to the above-mentioned ratio. However, it is preferable that the amount of radiation used to capture a radiographic image by the second radiation detector 20B is equal to or greater than 10% of the amount of radiation used to capture a radiographic image by the first radiation detector 20A in terms of diagnosis.

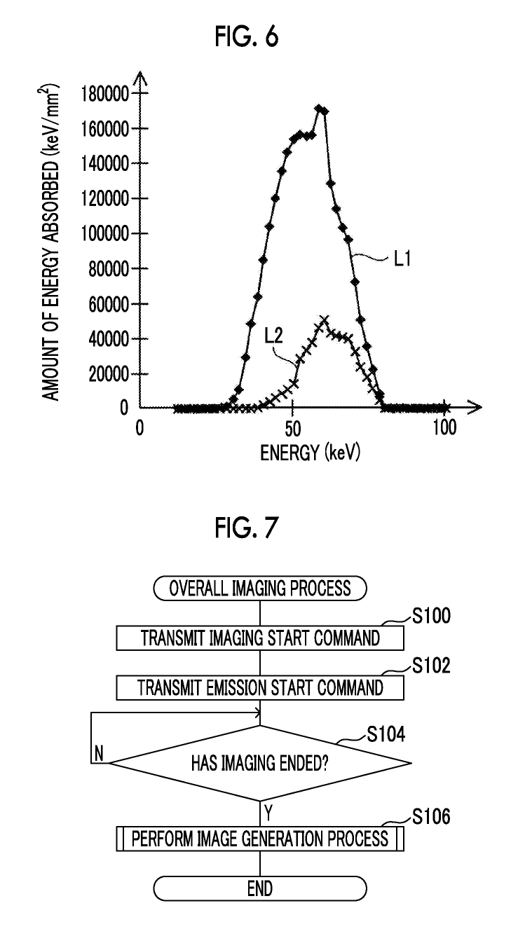

The radiation R is absorbed from a low-energy component. Therefore, for example, as illustrated in FIG. 6, the energy components of the radiation R that reaches the second radiation detector 20B do not include the low-energy components of the energy components of the radiation R that reaches the first radiation detector 20A. In FIG. 6, the vertical axis indicates the amount of radiation R absorbed per unit area and the horizontal axis indicates the energy of the radiation R in a case in which the tube voltage of the radiation source 14 is 80 kV. In addition, in FIG. 6, a solid line L1 indicates the relationship between the energy of the radiation R absorbed by the first radiation detector 20A and the amount of radiation R absorbed per unit area. In FIG. 6, a solid line L2 indicates the relationship between the energy of the radiation R absorbed by the second radiation detector 20B and the amount of radiation R absorbed per unit area.

Next, the operation of the radiography system 10 according to this embodiment will be described.

First, the operation of the console 18 will be described. FIG. 7 is a flowchart illustrating an example of the flow of an overall imaging process performed by the control unit 90 of the console 18. Specifically, the CPU 90A of the control unit 90 executes an overall imaging processing program to perform the overall imaging process illustrated in FIG. 7. The control unit 90 executes the overall imaging processing program to function as an example of a derivation unit according to the invention.

In this embodiment, the overall imaging process illustrated in FIG. 7 is performed in a case in which the control unit 90 of the console 18 acquires an imaging menu including, for example, the name of the subject W, an imaging part, and the emission conditions of the radiation R from the user through the operation unit 96. The control unit 90 may acquire the imaging menu from an external system, such as an RIS, or may acquire the imaging menu input by the user through the operation unit 96.

In Step S100 of FIG. 7, the control unit 90 of the console 18 transmits information included in the imaging menu as an imaging start command to the radiography apparatus 16 through the communication unit 98 and transmits the emission conditions of the radiation R to the radiation emitting apparatus 12 through the communication unit 98.

Then, in Step S102, the control unit 90 transmits a command to start the emission of the radiation R to the radiography apparatus 16 and the radiation emitting apparatus 12 through the communication unit 98. When receiving the emission conditions and the emission start command transmitted from the console 18, the radiation emitting apparatus 12 starts the emission of the radiation R according to the received emission conditions. The radiation emitting apparatus 12 may comprise an irradiation button. In this case, the radiation emitting apparatus 12 receives the emission conditions and the emission start command transmitted from the console 18 and starts the emission of the radiation R according to the received emission conditions in a case in which the irradiation button is pressed.

In the radiography apparatus 16, the first radiation detector 20A captures the first radiographic image and the second radiation detector 20B captures the second radiographic image, on the basis of the information in the imaging menu transmitted from the console 18, in response to the imaging start command, which will be described in detail below. In the radiography apparatus 16, the control units 58A and 58B perform various correction processes, such as offset correction and gain correction, for first radiographic image data indicating the captured first radiographic image and second radiographic image data indicating the captured second radiographic image, respectively, and store the corrected radiographic image data in the storage unit 64.

Then, in Step S104, the control unit 90 determines whether the capture of the radiographic images has ended in the radiography apparatus 16. A method for determining whether the capture of the radiographic images has ended is not particularly limited. For example, each of the control units 58A and 58B of the radiography apparatus 16 transmits end information indicating that imaging has ended to the console 18 through the communication unit 66. In a case in which the end information is received, the control unit 90 of the console 18 determines that the capture of the radiographic images has ended in the radiography apparatus 16.

For example, each of the control units 58A and 58B transmits the first radiographic image data and the second radiographic image data to the console 18 through the communication unit 66 after imaging ends. In a case in which the first radiographic image data and the second radiographic image data are received, the control unit 90 determines that the capture of the radiographic images by the radiography apparatus 16 has ended. In addition, in a case in which the first radiographic image data and the second radiographic image data are received, the console 18 stores the received first radiographic image data and the received second radiographic image data in the storage unit 92.

In a case in which the capture of the radiographic images by the radiography apparatus 16 has not ended, the determination result in Step S104 is “No” and the control unit 90 waits until the capture of the radiographic images by the radiography apparatus 16 ends. On the other hand, in a case in which the capture of the radiographic images by the radiography apparatus 16 has ended, the determination result in Step S104 is “Yes” and the control unit 90 proceeds to Step S106.

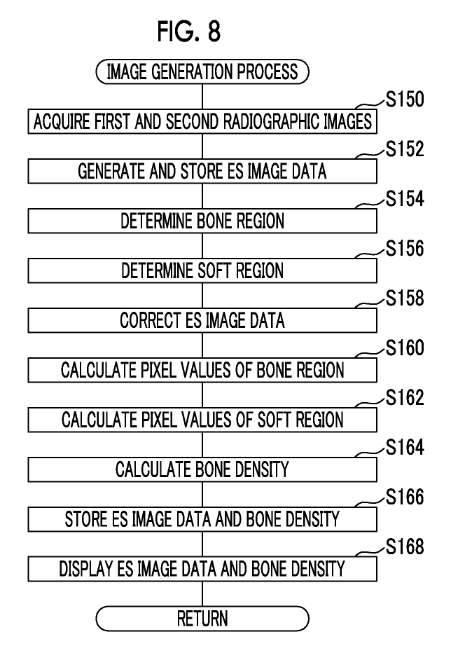

In Step S106, the control unit 90 performs an image generation process illustrated in FIG. 8 and ends the overall imaging process.

Next, the image generation process performed in Step S106 of the overall imaging process (see FIG. 7) will be described with reference to FIG. 8.

In Step S150 of FIG. 8, the control unit 90 of the console 18 acquires the first radiographic image data and the second radiographic image data. In a case in which the first radiographic image data and the second radiographic image data have been stored in the storage unit 92, the control unit 90 reads and acquires the first radiographic image data and the second radiographic image data from the storage unit 92. In a case in which the first radiographic image data and the second radiographic image data have not been stored in the storage unit 92, the control unit 90 acquires the first radiographic image data from the first radiation detector 20A and acquires the second radiographic image data from the second radiation detector 20B.

Then, in Step S152, the control unit 90 generates image data indicating an energy subtraction image, using the first radiographic image data and the second radiographic image data. Hereinafter, the energy subtraction image is referred to as an “ES image” and the image data indicating the energy subtraction image is referred to as “ES image data”.

In this embodiment, the control unit 90 subtracts image data obtained by multiplying the first radiographic image data by a predetermined coefficient from image data obtained by multiplying the second radiographic image data by a predetermined coefficient for each corresponding pixel. The control unit 90 generates ES image data indicating an ES image in which soft tissues have been removed and bone tissues have been highlighted, using the subtraction. A method for determining the corresponding pixels of the first radiographic image data and the second radiographic image data is not particularly limited. For example, the amount of positional deviation between the first radiographic image data and the second radiographic image data, which are captured by the radiography apparatus 16 in a state in which a marker is put in advance, may be calculated from the difference between the positions of the marker in the first radiographic image data and the second radiographic image data. Then, the corresponding pixels of the first radiographic image data and the second radiographic image data may be determined on the basis of the calculated amount of positional deviation.

In this case, for example, the amount of positional deviation between the first radiographic image data and the second radiographic image data, which are obtained by capturing the image of both the subject W and the marker when the image of the subject W is captured, may be calculated from the difference between the positions of the marker in the first radiographic image data and the second radiographic image data. In addition, for example, the amount of positional deviation between the first radiographic image data and the second radiographic image data may be calculated on the basis of the structure of the subject W in the first radiographic image data and the second radiographic image data obtained by capturing the image of the subject W.

Then, in Step S154, the control unit 90 determines a bone tissue region (hereinafter, referred to as a “bone region”) in the ES image that is indicated by the ES image data generated in Step S152. In this embodiment, for example, the control unit 90 estimates the approximate range of the bone region on the basis of the imaging part included in the imaging menu. Then, the control unit 90 detects pixels that are disposed in the vicinity of the pixels, of which the differential values are equal to or greater than a predetermined value, as the pixels forming the edge (end) of the bone region in the estimated range to determine the bone region.

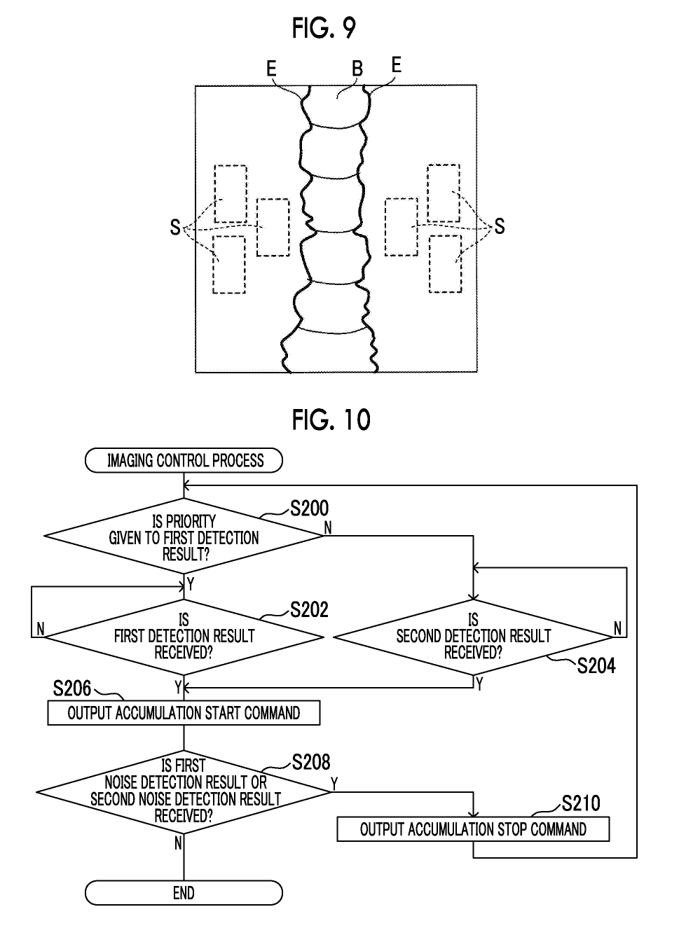

For example, as illustrated in FIG. 9, in Step S154, the control unit 90 detects the edge E of a bone region B and determines a region in the edge E as the bone region B. For example, FIG. 9 illustrates an ES image in a case in which the image of a backbone part of the upper half of the body of the subject W is captured.

A method for determining the bone region B is not limited to the above-mentioned example. For example, the control unit 90 displays the ES image that is indicated by the ES image data generated in Step S152 on the display unit 94. The user designates the edge E of the bone region B in the ES image displayed on the display unit 94 through the operation unit 96. Then, the control unit 90 may determine a region in the edge E designated by the user as the bone region B.

The control unit 90 may display an image in which the ES image and the edge E detected in Step S154 overlap each other on the display unit 94. In this case, in a case in which it is necessary to correct the edge E displayed on the display unit 94, the user corrects the position of the edge E through the operation unit 96. Then, the control unit 90 may determine a region in the edge E corrected by the user as the bone region B.

Then, in Step S156, the control unit 90 determines a soft tissue region (hereinafter, referred to as a “soft region”) in the ES image that is indicated by the ES image data generated in Step S152. In this embodiment, for example, the control unit 90 determines a region, which is other than the bone region B and has a predetermined area including pixels that are separated from the edge E by a distance corresponding to a predetermined number of pixels in a predetermined direction, as the soft region. For example, as illustrated in FIG. 9, in Step S156, the control unit 90 determines a plurality of (in the example illustrated in FIG. 9, six) soft regions S.

The predetermined direction and the predetermined number of pixels may be predetermined by, for example, experiments using the actual radiography apparatus 16 according to the imaging part. The predetermined area may be predetermined or may be designated by the user. In addition, for example, the control unit 90 may determine, as the soft region S, the pixels with pixel values in a predetermined range having the minimum pixel value (a pixel value corresponding to a position where the body thickness of the subject W is the maximum except the bone region B) as the lower limit in the ES image data. In addition, it goes without saying that the number of soft regions S determined in Step S156 is not limited to that illustrated in FIG. 9.

Then, in Step S158, the control unit 90 corrects the ES image data generated in Step S152 such that a variation in the ES image in each imaging operation is within an allowable range. In this embodiment, for example, the control unit 90 performs a correction process of removing image blur in the entire frequency band of the ES image data. The image data corrected in Step S158 is used to calculate bone density in a process from Step S160 to Step S164 which will be described below. Therefore, hereinafter, the corrected image data is referred to as “dual-energy X-ray absorptiometry (DXA) image data”.

Then, in Step S160, the control unit 90 calculates an average value A1 of the pixel values of the bone region B in the D×A image data. Then, in Step S162, the control unit 90 calculates an average value A2 of the pixel values of all of the soft regions S in the D×A image data. Here, in this embodiment, for example, the control unit 90 performs weighting such that the soft region S which is further away from the edge E has a smaller pixel value and calculates the average value A2. Before the average values A1 and A2 are calculated in Step S160 and Step S162, respectively, abnormal values of the pixel values of the bone region B and the pixel values of the soft region S may be removed by, for example, a median filter.

Then, in Step S164, the control unit 90 calculates the bone density of the imaging part of the subject W. In this embodiment, for example, the control unit 90 calculates the difference between the average value A1 calculated in Step S160 and the average value A2 calculated in Step S162. In addition, the control unit 90 multiplies the calculated difference by a conversion coefficient for converting the pixel value into bone mass [g] to calculate the bone mass. Then, the control unit 90 divides the calculated bone mass by the area [cm²] of the bone region B to calculate bone density [g/cm²]. The conversion coefficient may be predetermined by, for example, experiments using the actual radiography apparatus 16 according to the imaging part.

Then, in Step S166, the control unit 90 stores the ES image data generated in Step S152 and the bone density calculated in Step S164 in the storage unit 92 so as to be associated with information for identifying the subject W. In addition, for example, the control unit 90 may store the ES image data generated in Step S152, the bone density calculated in Step S164, the first radiographic image data, and the second radiographic image data in the storage unit 92 so as to be associated with the information for identifying the subject W.

Then, in Step S168, the control unit 90 displays the ES image indicated by the ES image data generated in Step S152 and the bone density calculated in Step S164 on the display unit 94 and then ends the image generation process.

Next, the operation of the radiography apparatus 16 according to this embodiment will be described.

As described above, when the radiography apparatus 16 according to this embodiment receives an imaging start command from the console 18, the first radiation detector 20A captures the first radiographic image and the second radiation detector 20B captures the second radiographic image under the control of the integrated control unit 71.

FIG. 10 is a flowchart illustrating an example of the flow of an imaging control process performed by the integrated control unit 71. Specifically, when the imaging start command is received from the console 18, the CPU 72 of the integrated control unit 71 executes an imaging control processing program that is stored in the ROM of the memory 74 in advance to perform the imaging control process illustrated in FIG. 10. The imaging control processing program is an example of a program including a radiography program according to the invention. In addition, the integrated control unit 71 executes an imaging processing program to function as an example of a specification unit according to the invention and to make the radiography apparatus 16 function as the radiography system 10 according to the invention.

In Step S200 of FIG. 10, the integrated control unit 71 determines which of the first detection result indicating the detection result of the start of the emission of the radiation R by the control unit 58A and the second detection result indicating the detection result of the start of the emission of the radiation R by the control unit 58B priority is given to. The determination in Step S200 may be performed only in a case in which the first detection result and the second detection result are different from each other.

In this embodiment, a method for determining the detection result with higher priority is not particularly limited. For example, in a case in which information indicating the priority of the detection results is set in the storage unit 76 of the integrated control unit 71 in advance, the set detection results may be read. In this case, as described above, since the amount of radiation R that reaches the second radiation detector 20B is less than the amount of radiation R that reaches the first radiation detector 20A, it is preferable that settings for giving priority to the first detection result obtained by the first radiation detector 20A are performed.

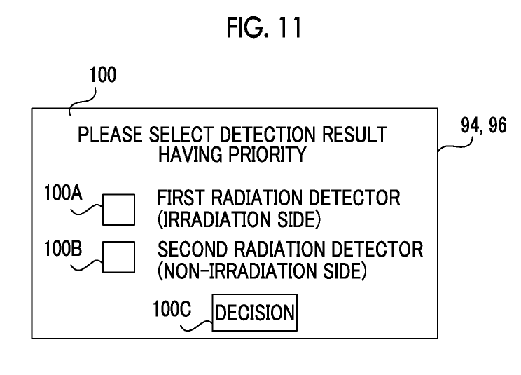

For example, as in the example illustrated in FIG. 11, the integrated control unit 71 may display a selection screen 100 that allows the user to select the detection result with priority on the display unit 94 of the console 18 through the communication unit 66 and perform the determination on the basis of the selection result of the user through the operation unit 96. According to the selection screen 100 illustrated in FIG. 11, in a case in which the user selects the first detection result obtained by the first radiation detector 20A, the user selects a selection box 100A through the operation unit 96. In a case in which the user selects the second detection result obtained by the second radiation detector 20B, the user selects a selection box 100B through the operation unit 96 and operates a decision button 100C through the operation unit 96. Then, the operation result is output from the console 18 to the radiography apparatus 16 through the communication unit 98. In this case, the operation unit 96 is an example of a detection result setting unit according to the invention.

In a case in which priority is given to the first detection result, the determination result in Step S200 is “Yes” and the process proceeds to Step S202. In Step S202, the integrated control unit 71 determines whether the first detection result has been received from the control unit 58A. In a case in which the first detection result has not been received, the determination result in Step S202 is “No” and the integrated control unit 71 waits until the first detection result is received. On the other hand, in a case in which the first detection result has been received, the determination result in Step S202 is “Yes” and the proceeds to Step S206.

In contrast, in a case in which priority is given to the second detection result, the determination result in Step S200 is “No” and the process proceeds to Step S204. In Step S204, the integrated control unit 71 determines whether the second detection result has been received from the control unit 58B. In a case in which the second detection result has not been received, the determination result in Step S204 is “No” and the integrated control unit 71 waits until the second detection result is received. On the other hand, in a case in which the second detection result has been received, the determination result in Step S204 is “Yes” and the proceeds to Step S206.

In Step S206, the integrated control unit 71 outputs an accumulation start command to the control unit 58A and the control unit 58B.

Then, in Step S208, the integrated control unit 71 determines whether a first noise detection result indicating the detection result of the generation of noise by the control unit 58A has been received from the control unit 58A or a second noise detection result indicating the detection result of the generation of noise by the control unit 58B has been received from the control unit 58B.

In a case in which at least one of the first noise detection result or the second noise detection result has been received, the determination result in Step S208 is “Yes” and the process proceeds to Step S210. In Step S210, the integrated control unit 71 outputs an accumulation stop command to the control unit 58A and the control unit 58B, returns to Step S200, and repeatedly performs the process in Steps S200 to S208.

On the other hand, in a case in which neither the first noise detection result nor the second noise detection result has been received after a predetermined period of time has elapsed, the determination result in Step S208 is “No” and the integrated control unit 71 ends the imaging control process. The predetermined period of time in this step is not particularly limited. An example of the predetermined period of time is a charge accumulation period in the radiation detector 20, which will be described in detail below.

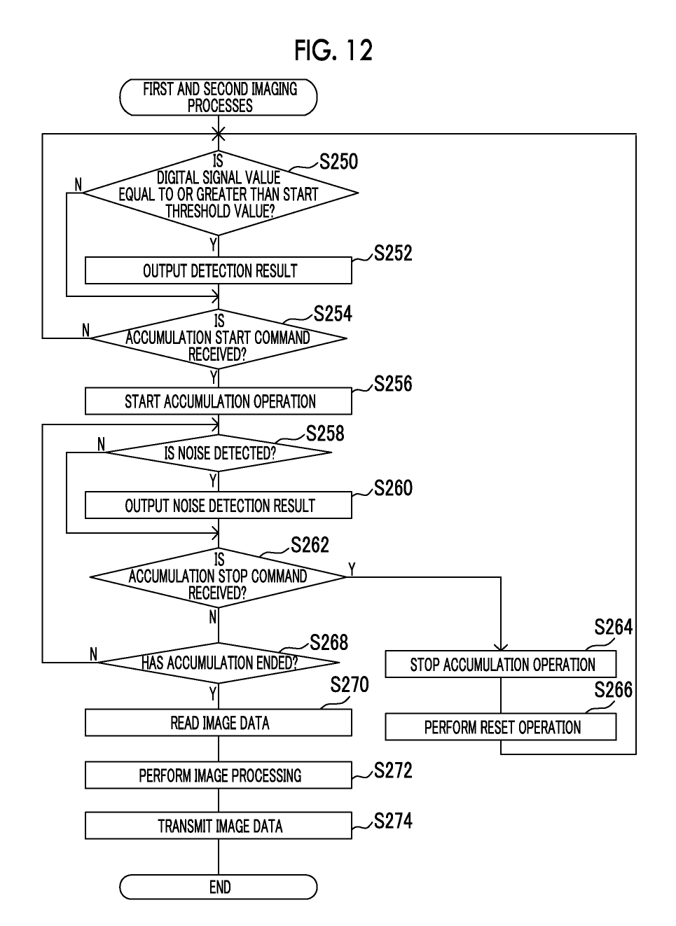

FIG. 12 is a flowchart illustrating an example of the flow of a first imaging process performed by the control unit 58A and an example of the flow of a second imaging process performed by the control unit 58B in the radiography apparatus 16. Specifically, when an imaging start command is received from the console 18, the CPU 60 of the control unit 58A executes a first imaging processing program that is stored in the ROM of the memory 62 in advance to perform the first imaging process illustrated in FIG. 12. In addition, when the imaging start command is received from the console 18, the CPU 60 of the control unit 58B executes a second imaging processing program that is stored in the ROM of the memory 62 in advance to perform the second imaging process illustrated in FIG. 12.

In Step S250 of FIG. 12, the control unit 58A determines whether the value of the first digital signal is equal to or greater than a predetermined start threshold value for detecting the start of the emission of the radiation R. Until image data is read in Step S270 which will be described below or until a reset operation is performed in Step S266, all of the thin film transistors 33C of the pixels 32 in the first radiation detector 20A are in an off state. However, as described above, a first electric signal corresponding to the charge which is read from the pixel 32B for detecting radiation regardless of a switching state is transmitted through the data line 36, is converted into the first digital signal by the signal processing unit 54A, and is output to the control unit 58A.

In a case in which the value of the first digital signal is equal to or greater than the predetermined start threshold value for detecting the start of the emission of the radiation R, the determination result in Step S250 is “Yes” and the process proceeds to Step S252. In Step S252, the control unit 58A outputs the first detection result indicating that the start of the emission of the radiation R has been detected to the integrated control unit 71 and proceeds to Step S254.

On the other hand, in a case in which the value of the first digital signal is less than the start threshold value in Step S250, the determination result is “No” and the process proceeds to Step S254. As such, the control unit 58A according to this embodiment uses a method that detects the time when the value of the first digital signal is equal to or greater than the start threshold value as the time when the emission of the radiation R has started. However, a method for detecting the time when the emission of the radiation R has started is not limited thereto. For example, the time when the value of the first digital signal is greater than the start threshold value may be detected as the time when the emission of the radiation R has started or the time when a variation in the first digital signal per unit time is equal to or greater than a predetermined start threshold value may be detected as the time when the emission of the radiation R has started.

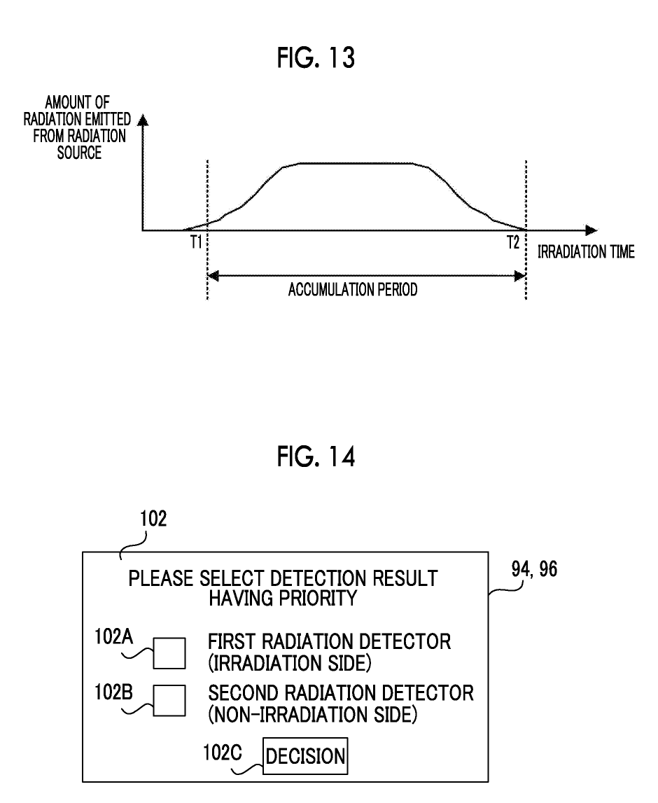

In this embodiment, the time when the emission of the radiation R has started is an example of a predetermined time related to the emission of radiation according to the invention. As in the example illustrated in FIG. 13, the amount of radiation R emitted from the radiation source 14 of the radiation emitting apparatus 12 varies depending on the irradiation time. In the radiography apparatus 16 according to this embodiment, a period from a time T1 to a time T2 illustrated in FIG. 13 is used as an accumulation period for which the above-mentioned accumulation operation is performed, according to the amount of radiation R that is emitted from the radiation source 14 to the radiography apparatus 16. Therefore, the time T1 is detected the time when the emission of the radiation R has started. Thus, the time when the radiation source 14 actually starts to emit the radiation R is different from the time when the radiography apparatus 16 starts to be irradiated with the radiation R. In addition, for example, the time T1 is determined in terms of an error in the detection of time.

In Step S254, the control unit 58A determines whether an accumulation start command has been received from the integrated control unit 71. In a case in which the accumulation start command has not been received, the determination result in Step S254 is “No” and the control unit 58A returns to Step S250. In a case in which the process proceeds to Step S254 after Step S252 and the accumulation start command has not been received, the control unit 58A may not return to Step S250 and wait until the accumulation start command is received. On the other hand, in a case in which the accumulation start command has been received, the determination result in Step S254 is “Yes” and the control unit 58A proceeds to Step S256.

In Step S256, the control unit 58A starts an accumulation operation. When the accumulation operation starts, the first radiation detector 20A proceeds to the accumulation period for which charge generated by the emitted radiation R is accumulated in the pixel 32. Specifically, the control unit 58A controls the gate line driver 52A such that an off signal is output from the gate line driver 52A to each gate line 34 of the first radiation detector 20A. Then, each thin film transistor 33C connected to each gate line 34 is turned off. As described above, after the accumulation operation starts, an electric signal corresponding to the charge that is read from the pixel 32B for detecting radiation is transmitted through the data line 36, is converted into the first digital signal by the signal processing unit 54A, and is output to the control unit 58A.

Then, in Step S258, the control unit 58A determines whether the inclusion of noise in the first digital signal has been detected. A method for detecting the inclusion of noise in the first digital signal in the control unit 58A is not particularly limited. Noise generated in the radiation detector 20 is disclosed in, for example, JP2014-023957A and a noise detection method disclosed in JP2014-023957A may be applied to this embodiment.

For example, in some cases, charge which will be noise is generated in the sensor unit 33A due to disturbance, such as impact and electromagnetic waves, particularly, vibration. An electric signal caused by noise (charge) that is generated due to disturbance has the characteristic that it is different from an electric signal caused by charge that is generated by irradiation with the radiation R in a general radiographic image. Particularly, the electric signals are different in a variation over time. For example, in a case in which noise is included, charge flows in the opposite direction. As a result, the polarity of the electric signal is likely to be opposite to the general polarity. In addition, in a case in which noise is included, a waveform indicating a variation in the electric signal over time has an amplitude.

In this embodiment, after the accumulation operation starts in Step S256, the control unit 58A detects whether noise has been generated, on the basis of whether a variation in a digital signal over time within a predetermined detection period has the above-mentioned characteristic of noise. As a detailed detection method, for example, the following methods are used: a method that detects whether noise has been generated, on the basis of whether a digital signal has a polarity opposite to the general polarity; a method that detects whether noise has been generated, on the basis of whether a gradient is reduced when differentiation (for example, first-order differentiation or second-order differentiation) is performed for a digital signal output within a predetermined period, for example, a method that differentiates the digital signal and detects that no noise has been generated in a case in which the gradient is substantially constant or is expected to gradually increase; and a method that detects whether noise has been generated, using a noise determination threshold value. In addition, it is preferable that a combination of a plurality of kinds of detection methods is used in order to increase the accuracy of detecting noise.

In a case in which the inclusion of noise in the first digital signal has been detected, the determination result in Step S258 is “Yes” and the control unit 58A proceeds to Step S260. In Step S260, the control unit 58A outputs the first noise detection result indicating that the inclusion of noise has been detected to the integrated control unit 71 and proceeds to Step S262.

On the other hand, in a case in which the inclusion of noise in the first digital signal has not been detected in Step S258, the determination result is “No” and the control unit 58A proceeds to Step S262.

Then, in Step S262, the control unit 58A determines whether an accumulation stop command has been received from the integrated control unit 71. In a case in which the accumulation stop command has been received, the determination result in Step S262 is “Yes” and the control unit 58A proceeds to Step S264.

In Step S264, the control unit 58A stops the operation of accumulating charge in the pixel 32. Then, in Step S266, the control unit 58A performs a reset operation of resetting the charge accumulated in the pixel 32 and returns to Step S250. Specifically, the control unit 58A controls the gate line driver 52A such that an on signal is output from the gate line driver 52A to each gate line 34 of the first radiation detector 20A. Then, each thin film transistor 33C connected to each gate line 34 is turned on and the charge accumulated in the capacitor 33B is output to the data line 36.

The period for which the reset operation is performed is a dead period (non-detection period) for which the start of the emission of the radiation R is not detected. Therefore, it is preferable to perform the reset operation for a plurality of gate lines 34 at the same time in order to shorten the dead period. In addition, during the reset operation, a command to stop the emission of the radiation R may be output to the radiation emitting apparatus 12 through the communication unit 66.

In contrast, in a case in which the accumulation stop command has not been received in Step S262, the determination result is “No” and the process proceeds to Step S268. In a case in which the process proceeds to Step S262 after Step S260 and the accumulation start command has not been received, the control unit 58A may wait until the accumulation start command is received, without proceeding to Step S268.

In Step S268, the control unit 58A determines whether to end the accumulation of charge. A method for determining whether to end the accumulation of charge is not particularly limited. For example, in a case in which a predetermined accumulation period has elapsed since the accumulation start command has been received, the control unit 58A may determine to end the accumulation of charge. In this case, in a case in which the predetermined accumulation period has not elapsed, the determination result in Step S268 is “No” and the process returns to Step S258. On the other hand, in a case in which the predetermined accumulation period has elapsed, the determination result in Step S268 is “Yes” and the process proceeds to Step S270.

Then, in Step S270, the control unit 58A ends the accumulation operation, proceeds to a reading period for which the charge accumulated in the pixel 32 is read, starts a reading operation, and controls the gate line driver 52A such that an on signal is sequentially output from the gate line driver 52A to each gate line 34 of the first radiation detector 20A. Then, the lines of the thin film transistors 33C connected to each gate line 34 are sequentially turned on and charge accumulated in each line of the capacitors 33B sequentially flows as an electric signal to each data line 36. Specifically, charge accumulated in the capacitors 33B of the pixels 32A for capturing a radiographic image flows as an electric signal to the data line 36. Then, the electric signal that has flowed to each data line 36 is converted into digital image data by the signal processing unit 54A, is output from the control unit 58A to the image memory 56A, and is then stored in the image memory 56A.

Then, in Step S272, the control unit 58A performs image processing including various correction processes, such as offset correction and gain correction, for the image data stored in the image memory 56A in Step S270. Then, in Step S274, the control unit 58A transmits the image data (first radiographic image data) processed in Step S272 to the integrated control unit 71 and ends the first imaging process.

As illustrated in FIG. 12, the first imaging process and the second imaging process are the same process. In the second imaging process, the control unit 58B may replace the control unit 58A, the second digital signal may replace the first digital signal, the second detection result may replace the first detection result, and the second noise detection result may replace the first noise detection result. In addition, the gate line driver 52B may replace the gate line driver 52A, the signal processing unit 54B may replace the signal processing unit 54A, and the image memory 56B may replace the image memory 56A. Therefore, the description of the components will not be repeated.

As described above, since the amount of radiation R that reaches the second radiation detector 20B is less than the amount of radiation R that reaches the first radiation detector 20A, the start threshold value used by the first radiation detector 20A may be different from the start threshold value used by the second radiation detector 20B.

As described above, the radiography system 10 according to this embodiment comprises: the radiography apparatus 16 comprising the first radiation detector 20A in which a plurality of pixels 32, each of which includes the sensor unit 33A that generates a larger amount of charge as it is irradiated with a larger amount of radiation R, are two-dimensionally arranged and the second radiation detector 20B which is provided so as to be stacked on the side of the first radiation detector 20A from which the radiation R is transmitted and emitted and in which a plurality of pixels 32, each of which includes the sensor unit 33A that generates a larger amount of charge as it is irradiated with a larger amount of radiation R, are two-dimensionally arranged; and the integrated control unit 71 that specifies a predetermined time related to the emission of the radiation R, on the basis of a first detection result that is a detection result of a predetermined time related to the emission of the radiation R using a first electric signal (first digital signal) which is obtained by converting charge generated in the pixels 32 of the first radiation detector 20A and of which the level increases as the amount of charge generated increases and a second detection result that is a detection result of a predetermined time related to the emission of the radiation R using a second electric signal (second digital signal) which is obtained by converting charge generated in the pixels 32 of the second radiation detector 20B and of which the level increases as the amount of charge generated increases.

In the radiography apparatus 16 according to this embodiment, the amount of radiation that reaches the second radiation detector 20B is less than the amount of radiation that reaches the first radiation detector 20A. Therefore, in some cases, the first detection result which is the detection result of the predetermined time related to the emission of the radiation R using the first digital signal output from the first radiation detector 20A is different from the second detection result which is the detection result of the predetermined time related to the emission of the radiation R using the second digital signal output from the second radiation detector 20B.

In the radiography apparatus 16 according to this embodiment, the integrated control unit 71 specifies the time when the emission of the radiation R starts, using the first detection result and the second detection result, specifically, one of the first detection result and the second detection result which has higher priority.

Therefore, according to the radiography system 10 of each of the above-described embodiments, it is possible to appropriately detect the emission of the radiation R even when the amount of radiation R emitted to the second radiation detector is less than the amount of radiation R emitted to the first radiation detector.

In this embodiment, the case in which the control unit 58A and the control unit 58B detects the time when the emission of the radiation R starts as the predetermined time related to the emission of the radiation R has been described. However, the invention is not limited thereto. For example, the control unit 58A and the control unit 58B may detect the time when the emission of the radiation R is stopped like the time T2 illustrated in FIG. 13. In this case, for example, the control unit 58A and the control unit 58B may compare the value of the above-mentioned digital signal with a predetermined stop threshold value for detecting the stop of the emission of the radiation R and may determine that it is time to stop the emission of the radiation R in a case in which the value of the digital signal is less than the stop threshold value. In addition, as such, in a case in which the time when the emission of the radiation R is stopped is detected, the control unit 58A and the control unit 58B may end the accumulation of charge in the pixel 32 and may proceed to the reading period.

In this embodiment, the case in which an indirect-conversion-type radiation detector that converts radiation into light and converts the converted light into charge is applied to both the first radiation detector 20A and the second radiation detector 20B has been described. However, the invention is not limited thereto. For example, a direct-conversion-type radiation detector that directly converts radiation into charge may be applied to at least one of the first radiation detector 20A or the second radiation detector 20B.

In the radiography apparatus 16 according to this embodiment, the aspect in which the pixels 32 comprise the pixel 32B for detecting radiation in which the thin film transistor 33C is short-circuited and the predetermined time related to the emission of the radiation R is detected using the electric signal generated by charge output from the pixel 32B for detecting radiation has been described. However, the invention is not limited thereto. For example, a technique disclosed in JP2014-023957A can be applied to detect the predetermined time related to the emission of the radiation R. Specifically, for example, all of the pixels 32 connected to a specific gate line 34 may be used as the pixels 32B for detecting radiation. In this case, the pixel 32B for detecting radiation comprises a thin film transistor 33C that is not short-circuited. In a case in which the predetermined time related to the emission of the radiation R is detected, the control unit 58A and the control unit 58B control the gate line driver 52A and the gate line driver 52B such that the on signals are output from the gate line driver 52A and the gate line driver 52B to the gate lines 34 connected to the pixels 32B for detecting radiation in the first radiation detector 20A and the second radiation detector 20B, respectively. In addition, for example, a first electric signal output from a sensor that is provided so as to correspond to the first radiation detector 20A and outputs the first electric signal of which the level increases as the amount of radiation R detected increases and a second electric signal output from a sensor that is provided so as to correspond to the second radiation detector 20B and outputs the second electric signal of which the level increases as the amount of radiation R detected increases may be used.

In this embodiment, in a case in which at least one of the first noise detection result or the second noise detection result indicates that the generation of noise has been detected, the integrated control unit 71 directs the control unit 58A and the control unit 58B to stop the accumulation of charge in each pixel 32. However, the invention is not limited thereto. For example, similarly to the first detection result and the second detection result, in a case in which the first noise detection result and the second noise detection result are different from each other, priority may be given to one of the noise detection results. In this case, for example, in a case in which information indicating which of the noise detection results has priority is set in the storage unit 76 of the integrated control unit 71 in advance, the set noise detection result may be read. In this case, as described above, since the amount of radiation R that reaches the second radiation detector 20B is less than the amount of radiation R that reaches the first radiation detector 20A, it is preferable that settings for giving priority to the first noise detection result obtained by the first radiation detector 20A are performed.

For example, as in the example illustrated in FIG. 14, the integrated control unit 71 may display a selection screen 102 that allows the user to select the noise detection result having priority on the display unit 94 of the console 18 through the communication unit 66 and may perform determination on the basis of the selection result selected by the user through the operation unit 96. According to the selection screen 102 illustrated in FIG. 14, in a case in which the user selects the first noise detection result obtained by the first radiation detector 20A, the user selects a selection box 102A through the operation unit 96. In a case in which the user selects the second noise detection result obtained by the second radiation detector 20B, the user selects a selection box 102B through the operation unit 96 and operates a decision button 102C through the operation unit 96. Then, the operation result is output from the console 18 to the radiography apparatus 16 through the communication unit 98. In this case, the operation unit 96 is an example of a noise detection result setting unit according to the invention.

In this embodiment, as described above, the case in which the control unit 58A detects noise included in the first digital signal, using the first digital signal, and the control unit 58B detects noise included in the second digital signal, using the second digital signal has been described. However, a structure for detecting noise is not limited thereto.

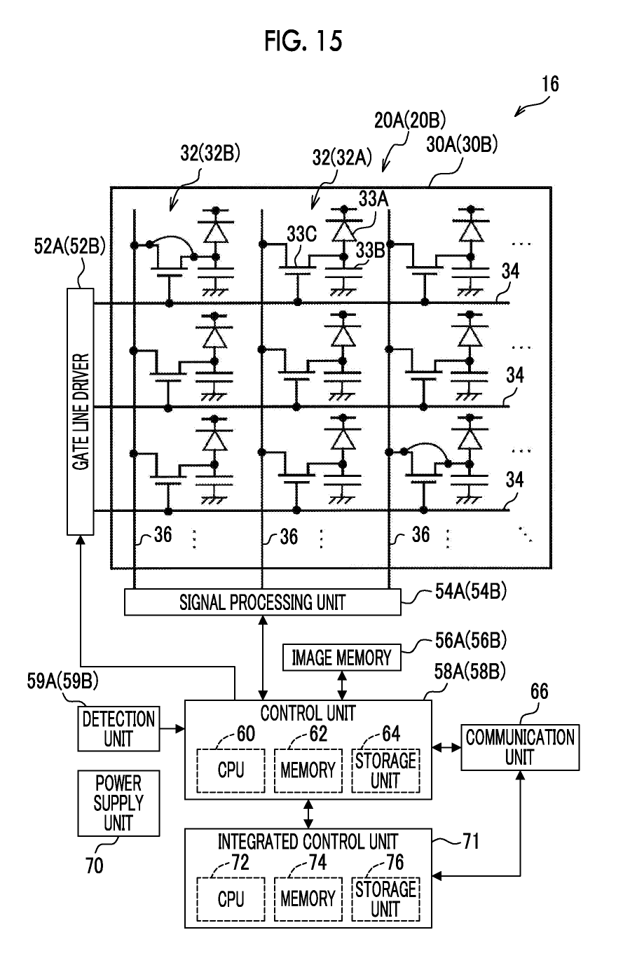

For example, as illustrated in FIG. 15, the radiography apparatus 16 may further comprise a detection unit 59A and a detection unit 59B. The control unit 58A may detect that noise is included in the first digital signal, using the detection result of the detection unit 59A, and the control unit 58B may detect that noise is included in the second digital signal, using the detection result of the detection unit 59B.

The detection unit 59A is not particularly limited as long as it can detect at least one of an impact or electromagnetic waves applied from the outside to the first radiation detector 20A. In addition, the detection unit 59B is not particularly limited as long as it can detect at least one of impact or electromagnetic waves applied from the outside to the second radiation detector 20B. In this case, the term “outside” may be the outside of each of the first radiation detector 20A and the second radiation detector 20B or may be one of the inside and the outside of the radiography apparatus 16. In this case, the detection unit 59A is an example of a first detection unit according to the invention and the detection unit 59B is an example of a second detection unit according to the invention.

For example, an impact sensor that directly detects an impact or an electromagnetic wave sensor that detects electromagnetic waves may be used as the detection unit 59A and the detection unit 59B. In a case in which the detection unit 59A and the detection unit 59B are impact sensors, an example of the detection unit 59A and the detection unit 59B is an acceleration sensor. In a case in which the impact sensor is used, it is preferable that the impact sensor is electro-magnetically shielded.

For example, in a case in which the detection unit 59A is the impact sensor, when detecting that the generation of an impact on the first radiation detector 20A is detected, the detection unit 59A outputs a signal indicating the generation of the impact as the detection result to the control unit 58A. In a case in which the detection unit 59B is the impact sensor, similarly, when detecting that the generation of an impact on the second radiation detector 20B is detected, the detection unit 59B outputs a signal indicating the generation of the impact as the detection result to the control unit 58B.

Then, the control unit 58A detects whether noise is included in the first digital signal, using the detection result of the detection unit 59A, specifically, on the basis of whether the signal indicating the generation of the impact is input from the detection unit 59A, in Step S258 (see FIG. 12) of the first imaging process. Similarly, the control unit 58B detects whether noise is included in the second digital signal, using the detection result of the detection unit 59B, specifically, on the basis of whether the signal indicating the generation of the impact is input from the detection unit 59B, in Step S258 (see FIG. 12) of the second imaging process.

In this embodiment, the case in which the irradiation side sampling radiation detectors in which the radiation R is incident from the side of the TFT substrates 30A and 30B are applied to the first radiation detector 20A and the second radiation detector 20B, respectively, has been described. However, the invention is not limited thereto. For example, a so-called penetration side sampling (PSS) radiation detector in which the radiation R is incident from the side of the scintillator 22A or 22B may be applied to at least one of the first radiation detector 20A or the second radiation detector 20B.

In this embodiment, the case in which the radiography apparatus 16 is controlled by three control units (control units 58A, 58B, and 71) has been described. However, the invention is not limited thereto. For example, one of the control unit 58A and the control unit 58B may have the functions of the integrated control unit 71 or the integrated control unit 71 may have the functions of the control unit 58A and the control unit 58B. In addition, the radiography apparatus 16 may be controlled by one control unit.

In this embodiment, for example, the case in which the integrated control unit 71 of the radiography apparatus 16 has the functions of the specification unit according to the invention has been described. However, the invention is not limited thereto. For example, the control unit 90 of the console 18 may execute the imaging control processing program (see FIG. 10) to function as an example of the specification unit according to the invention.

In this embodiment, the case in which bone density is derived using the first radiographic image and the second radiographic image has been described. However, the invention is not limited thereto. For example, bone mineral content or both bone density and bone mineral content may be derived using the first radiographic image and the second radiographic image.

In this embodiment, the aspect in which the overall imaging processing program is stored (installed) in the ROM 90B in advance, the imaging control processing program is stored in the memory 74 in advance, the first imaging processing program is stored in the memory 62 in advance, and the second imaging processing program is stored in the memory 62 in advance has been described. However, the invention is not limited thereto. Each of the overall imaging processing program, the imaging control processing program, the first imaging processing program, and the second imaging processing program may be recorded in a recording medium, such as a compact disk read only memory (CD-ROM), a digital versatile disk read only memory (DVD-ROM), or a universal serial bus (USB) memory, and then provided. In addition, each of the overall imaging processing program, the imaging control processing program, the first imaging processing program, and the second imaging processing program may be downloaded from an external apparatus through a network.

What is claimed is:

1. A radiography system comprising:
a radiography apparatus comprising a first radiation detector in which a plurality of pixels, each of which includes a conversion element that generates a larger amount of charge as it is irradiated with a larger amount of radiation, are two-dimensionally arranged and a second radiation detector which is provided at a side of the first radiation detector from which the radiation is transmitted and emitted and in which a plurality of pixels, each of which includes a conversion element that generates a larger amount of charge as it is irradiated with a larger amount of radiation, are two-dimensionally arranged; and
a specification unit that specifies a predetermined time related to the emission of the radiation, on the basis of a first detection result that is a detection result of a predetermined time related to the emission of the radiation using a first electric signal which is obtained by converting charge generated in the pixels of the first radiation detector and of which the level increases as the amount of charge increases and a second detection result that is a detection result of a predetermined time related to the emission of the radiation using a second electric signal which is obtained by converting charge generated in the pixels of the second radiation detector and of which the level increases as the amount of charge increases.

2. The radiography system according to claim 1,
wherein, in a case in which the first detection result and the second detection result are different from each other, the specification unit specifies the predetermined time related to the emission of the radiation on the basis of a predetermined detection result of the first and second detection results.

3. The radiography system according to claim 2,
wherein the predetermined detection result is the first detection result.

4. The radiography system according to claim 2, further comprising:
a detection result setting unit that sets the predetermined detection result.

5. The radiography system according to claim 1,
wherein the specification unit further specifies whether to continue to perform an operation of accumulating charge in the plurality of pixels of the first radiation detector and an operation of accumulating charge in the plurality of pixels of the second radiation detector, using a first noise detection result which is a detection result of noise included in the first electric signal and a second noise detection result which is a detection result of noise included in the second electric signal after the operation of accumulating charge in the plurality of pixels of the first radiation detector and the operation of accumulating charge in the plurality of pixels of the second radiation detector start.

6. The radiography system according to claim 5,
wherein the first noise detection result and the second noise detection result that the specification unit uses to specify whether to continue to perform the operation of accumulating charge in the plurality of pixels of the first radiation detector and the operation of accumulating charge in the plurality of pixels of the second radiation detector are a detection result of noise included in the first electric signal using the first electric signal and a detection result of noise included in the second electric signal using the second electric signal, respectively.

7. The radiography system according to claim 5, further comprising:
a first detection unit that detects at least one of an impact or an electromagnetic wave which is applied from the outside to the first radiation detector; and
a second detection unit that detects at least one of an impact or an electromagnetic wave which is applied from the outside to the second radiation detector,
wherein the first noise detection result and the second noise detection result that the specification unit uses to specify whether to continue to perform the operation of accumulating charge in the plurality of pixels of the first radiation detector and the operation of accumulating charge in the plurality of pixels of the second radiation detector are a detection result of noise included in the first electric signal using a detection result of the first detection unit and a detection result of noise included in the second electric signal using a detection result of the second detection unit, respectively.

8. The radiography system according to claim 5,
wherein, in a case in which the first noise detection result and the second noise detection result are different from each other, the specification unit specifies whether to continue to perform the operation of accumulating charge in the plurality of pixels of the first radiation detector and the operation of accumulating charge in the plurality of pixels of the second radiation detector, using a predetermined noise detection result of the first and second noise detection results.

9. The radiography system according to claim 8,
wherein the predetermined noise detection result is the first noise detection result.

10. The radiography system according to claim 8, further comprising:
a noise detection result setting unit that sets the predetermined noise detection result.

11. The radiography system according to claim 5,
wherein, in a case in which at least one of the first noise detection result or the second noise detection result indicates that noise has been detected, the specification unit specifies to stop the operation of accumulating charge in the plurality of pixels of the first radiation detector and the operation of accumulating charge in the plurality of pixels of the second radiation detector.

12. The radiography system according to claim 1,
wherein the specification unit specifies a time when the emission of the radiation starts as the predetermined time related to the emission of the radiation.

13. The radiography system according to claim 1,
wherein the radiography apparatus further comprises the specification unit.

14. The radiography system according to claim 1,
wherein each of the first radiation detector and the second radiation detector comprises a light emitting layer that is irradiated with radiation and emits light,
the plurality of pixels of each of the first radiation detector and the second radiation detector receive the light, generate the charge, and accumulate the charge, and
the light emitting layer of the first radiation detector and the light emitting layer of the second radiation detector have different compositions.

15. The radiography system according to claim 14,
wherein the light emitting layer of the first radiation detector includes CsI, and
the light emitting layer of the second radiation detector includes GOS.

16. The radiography system according to claim 1, further comprising:
a derivation unit that derives at least one of bone mineral content or bone density, using a first radiographic image captured by the first radiation detector and a second radiographic image captured by the second radiation detector.

17. A radiography method that is performed by a radiography apparatus comprising a first radiation detector in which a plurality of pixels, each of which includes a conversion element that generates a larger amount of charge as it is irradiated with a larger amount of radiation, are two-dimensionally arranged and a second radiation detector which is provided at a side of the first radiation detector from which the radiation is transmitted and emitted and in which a plurality of pixels, each of which includes a conversion element that generates a larger amount of charge as it is irradiated with a larger amount of radiation, are two-dimensionally arranged, the method comprising:
specifying a predetermined time related to the emission of the radiation, on the basis of a first detection result that is a detection result of a predetermined time related to the emission of the radiation using a first electric signal which is obtained by converting charge generated in the pixels of the first radiation detector and of which the level increases as the amount of charge increases and a second detection result that is a detection result of a predetermined time related to the emission of the radiation using a second electric signal which is obtained by converting charge generated in the pixels of the second radiation detector and of which the level increases as the amount of charge increases.

18. A non-transitory computer readable storage medium storing a radiography program that causes a computer to execute a process of controlling a radiography apparatus comprising a first radiation detector in which a plurality of pixels, each of which includes a conversion element that generates a larger amount of charge as it is irradiated with a larger amount of radiation, are two-dimensionally arranged and a second radiation detector which is provided at a side of the first radiation detector from which the radiation is transmitted and emitted and in which a plurality of pixels, each of which includes a conversion element that generates a larger amount of charge as it is irradiated with a larger amount of radiation, are two-dimensionally arranged, the process comprising:
specifying a predetermined time related to the emission of the radiation, on the basis of a first detection result that is a detection result of a predetermined time related to the emission of the radiation using a first electric signal which is obtained by converting charge generated in the pixels of the first radiation detector and of which the level increases as the amount of charge increases and a second detection result that is a detection result of a predetermined time related to the emission of the radiation using a second electric signal which is obtained by converting charge generated in the pixels of the second radiation detector and of which the level increases as the amount of charge increases.
